SEQUENCE LISTING (9 sequences)

SEQ ID NO: 1 - DNA, 711 bp, Homo sapiens
atggcggagccatcggcggccactcagtcccattccatctcctcgtcgtccttcggagccgagccgtccgcgcccggcggcggcgggagcccaggagcctgccccgccctggggacgaagagctgcagctcctcctgtgcggtgcacgatctgattttctggagagatgtgaagaagactgggtttgtctttggcaccacgctgatcatgctgctttccctggcagctttcagtgtcatcagtgtggtttcttacctcatcctggctcttctctctgtcaccatcagcttcaggatctacaagtccgtcatccaagctgtacagaagtcagaagaaggccatccattcaaagcctacctggacgtagacattactctgtcctcagaagctttccataattacatgaatgctgccatggtgcacatcaacagggccctgaaactcattattcgcctctttctggtagaagatctggttgactccttgaagctggctgtcttcatgtggctgatgacctatgttggtgctgtttttaacggaatcacccttctaattcttgctgaactgctcattttcagtgtcccgattgtctatgagaagtacaagacccagattgatcactatgttggcatcgcccgagatcagaccaagtcaattgttgaaaagatccaagcaaaactccctggaatcgccaaaaaaaaggcagaataa

SEQ ID NO: 2 - protein, 236 aa, Homo sapiens
MAEPSAATQSHSISSSSFGAEPSAPGGGGSPGACPALGTKSCSSSCAVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 3 - DNA, 32 bp, Homo sapiens
atatatggatccctcgctcgcgtagccatggc

SEQ ID NO: 4 - DNA, 32 bp, Homo sapiens
atatatgcggccgcgtttccatgtacttattc

SEQ ID NO: 5 - DNA, 32 bp, Homo sapiens
aaaaaggcagaagtacatggaaacgcggccgc

SEQ ID NO: 6 - DNA, 35 bp, Homo sapiens
ttccatgtactttctgccttttttttggcgattcc

SEQ ID NO: 7 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRPGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLINTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFPAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 8 - protein, 373 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 9 - protein, 199 aa, Homo sapiens
MDGQKKNWKDKVVDLLYWRDIKKTGVVFGASLFLLLSLTVFSIVSVTAYIALALLSVTISFRIYKGVIQAIQKSDEGHPFPAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

(19) United States
(12) Patent Application Publication
(10) Pub. No.: US 2004/0063161 A1
(43) Pub. Date: Apr. 1, 2004

(54) Compositions and method of treating Alzheimer's disease

(76) Inventors: Riqiang Yan, Kalamazoo, MI (US); Yifeng Lu, Portage, MI (US)

Correspondence Address: PHARMACIA & UPJOHN, 301 HENRIETTA ST, 0228-32-LAW, KALAMAZOO, MI, 49007, US

(21) Appl. No.: 10/408,967

(22) Filed: Apr. 8, 2003

Related U.S. Application Data
(60) Provisional application No. 60/373,284, filed on Apr. 17, 2002.

(51) Int. Cl. G01N 33/53; G01N 33/567; A61K 38/17; C07K 14/47

(52) U.S. Cl. 435/7.2; 514/12; 530/324

(57) ABSTRACT

The invention relates to compositions and methods for treating Alzheimer's Disease and other amyloidoses, to polypeptides that modulate BACE1 activity, and methods to identify agents for use in treating Alzheimer's Disease and other amyloidoses.

### CROSS-REFERENCE TO RELATED APPLICATIONS

This application claims the benefit of the following provisional application: U.S. provisional application Serial No. 60/373,284 filed on Apr. 17, 2002, under 35 USC 119(e)(i).

### FIELD OF THE INVENTION

The invention relates generally to compositions and methods for treating Alzheimer's disease and other amyloidosis, and particularly to polypeptides that modulate BACE1 activity and methods of identifying agents for use in treating Alzheimer's disease and other amyloidosis.

### BACKGROUND OF THE INVENTION

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by the presence of extracellular senile plaques and intracellular neurofibrillary tangles in the brains of affected individuals (Masters, C. L. et al., Proc. Natl. Acad. Sci. USA, 82:4245-4249 (1985)). While the plaques form primarily in particular parts of the brain—such as the hippocampus—in some cases they are also found in the walls of cerebral and meningeal blood vessels. (Delacourt, A. et al., Virchows Archiv.—A, Pathological Analomy & Histopathology, 411:199-204 (1987); and Masters, C. L. et al., EMBO Journal, 4:2757-2763 (1985)).

The senile plaques in AD were found to be composed predominantly of an aggregate of heterogeneous peptide fragments know as A beta (and also referred to in the art as amyloid beta peptide, beta amyloid peptide, beta amyloid protein, A beta peptide, A beta protein, or A4 protein). A beta peptide is a 39-43 amino acid protein that is a cleavage product of a much larger precursor protein called amyloid precursor protein (APP).

Several lines of evidence indicate that progressive cerebral deposition of beta-amyloid peptide plays a seminal role in the pathogenesis of AD and can precede cognitive symptoms by years or decades (See, for example, Selkoe, 1991, Neuron 6:487). Release of A beta from neuronal cells grown in culture and the presence of A beta in cerebrospinal fluid (CSF) of both normal individuals and AD patients has been demonstrated (See, for example, Seubert et al., 1992, Nature 359:325-327).

Strong evidence that amyloid beta protein deposition plays a critical role in the development of Alzheimer's disease came from the identification of familial Alzheimer's disease kindreds in which the Alzheimer's disease phenotype co-segregates with mutations from the amyloid precursor protein gene. (Younkin, S. G., Tohuku J. of Exper. Med., 174:217-223 (1994); and Matsumura, Y. et al., Neurology, 46:1721-1723 (1996)).

Amyloidogenic plaques and/or vascular amyloid angiopathy are also found to be associated with other disorders such as Trisomy 21 (Down's Syndrome), Hereditary Cerebral Hemorrhage, Cerebral Amyloid Angiopathy, and Sporadic Inclusion-body Myositis (the most common progressive muscle disease of older individuals) and other neurogenerative disorders.

Amyloid beta peptide, sometimes known as “beta amyloid peptide “A beta peptide,” “beta amyloid,” “A beta,” or “Aβ,” is derived by proteolysis of the amyloid precursor protein (APP). Several proteases called secretases are involved in the processing of APP. Cleavage of APP at the N-terminus of the A beta peptide by beta-secretase and at the C-terminus by one or more gamma-secretases constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

A membrane bound aspartyl protease named BACE1, Asp2, or memapsin 2, was identified as a beta-secretase, the enzyme responsible for processing of APP at the beta-secretase cleavage site to form A beta (Yan et al., 1999; Vassar et al., 1999; Hussain et al., 1999; Lin et al, 2000, Sinha et. al., 1999). BACE1 deficient mice almost completely block the production of A beta, suggesting that BACE1 is the principal cellular beta-secretase (Cai et al., 2001; Lou et al, 2001, Roberds et al., 2001). Endogenous BACE1 was found to localize predominantly in the later Golgi and TGN compartments where it cleaves APP to produce secreted APPb fragments and membrane bound C-terminal fragment CTF99 (Yan et al., 2001). CTF99 can be further processed by gamma-secretase to release amyloid peptides (A beta).

Because of the critical role of BACE1 in A beta production, it is believed that inhibition of this enzyme's activity is desirable for treating or delaying the onset of AD and other disorders associated with A beta deposits.

Proteins of the reticulon (RTN) family, also known as neutoendocrine-specific proteins (NSPs), are preferentially expressed in neuroendocrine tissues. These proteins are known to be associated with the endoplasmic reticulum. To date, four human reticulon genes (RTN1, RTN2, RTN3, RTN4) have been cloned. Moreira et al disclose a human RTN3 amino acid sequence of SEQ ID NO. 2 and RTN3 nucleotide sequence of the coding region of SEQ ID NO. 1. (E. F. Moreira, C. J. Jaworski, and I. R. Rodriguez, Cloning of a novel member of the reticulon gene family (RTN3): gene structure and chromosomal localization to 11q13. Genomics 58, 73-81 (1999)). Reticulon 4 (RTN4), also known as foocen or Nogo, is a homolog of RTN3. Three isoforms of RTN4 gene products have been identified, which are RTN4-A, RTN4-B and RTN4-C, also known as Nogo A, Nogo B, and Nogo C, respectively. The RTN4-B and RTN4-C are alternative splicing variants of RTN4-A. WO 00/31235 discloses amino acid sequences of the three RTN4 isoforms and the nucleic acid sequences encoding the RTN4 isoforms for rats and humans. WO 00/05364 and WO 01/3631 also disclose human RTN4-A amino acid sequence of SEQ ID NO. 7, human RTN4-B amino acid sequence of SEQ ID NO. 8, and human RTN4-C amino acid sequence of SEQ ID NO. 9. Reference to RTN4 herein includes all three isoforms of RTN4 polypeptides unless otherwise specified.

Prominent immunoreactivity of these proteins was detected in many brain regions, including cerebellum, superior colliculus, hippocampus, substantia nigra, and caudate putamen. The exact function of these reticulons is not known; however it has been suggested that they may play a role in vesicular formation, packaging of secretory products or regulation of intracellular Ca levels.

### BRIEF DESCRIPTION OF DRAWINGS

FIG. 1. Immunocomplex of BACE1. BACE1 was immunoprecipitated from HEK-293 cells transfected with HA-tagged BACE1 using anti-HA antibody. The eluted immunocomplex was resolved by 4-12% NUPAGE gel followed by Colloidal Blue staining. The bands that were indicated with arrowheads were confirmed as BACE1 and its degraded fragments by Western blot analysis.

### BRIEF DESCRIPTION OF SEQUENCE LISTING

SEQ ID No. 1: polynucleotide sequence of human RTN3

SEQ ID NO. 2: amino acid sequence of human RTN3

SEQ ID No. 3: polynucleotide sequence (PCR primer)

SEQ ID NO. 4: polynucleotide sequence (PCR primer)

SEQ ID No. 5: polynucleotide sequence (PCR primer)

SEQ ID NO. 6: polynucleotide sequence (PCR primer)

SEQ ID No. 7: amino acid sequence of human RTN4-A

SEQ ID NO. 8: amino acid sequence of human RTN4-B

SEQ ID NO. 9: amino acid sequence of human RTN4-C

### SUMMARY OF THE INVENTION

The present invention is based, in part, on the novel finding that RTN3 or RTN4 modulates the activity of BACE1. Thus, in one aspect, the invention provides a method of modulating BACE1 activity in a human and non-human animal by administration of an exogenous RTN3 or exogenous RTN4 polypeptide or administration of one or more agents that affect the expression or activity of endogenous RTN3 or RTN4.

The invention further provides recombinant polypeptides that are derived from RTN3 sequence and possess one or more function or biological activities of RTN3, polynucleotide sequences encoding the recombinant polypeptides, and method of making the recombinant polypeptides.

The invention further provides in vitro or in vivo methods to identify agents that modulate (1) the expression or activity of RTN3 or RTN4 or (2) the activity of BACE1.

The invention further provides agents for use in modulating the activity of BACE1 said agents including exogenous RTN3, exogenous RTN4 polypeptide, recombinant polypeptides of the invention, and agents that affect the expression or activity of endogenous RTN3 or RTN4.

The invention also provides methods of treating or delaying the onset of disorders associated with beta amyloid deposits in human or non-human animal said method comprising administration of an exogenous RTN3, exogenous RTN4 polypeptide, recombinant polypeptides of the invention, an agents that affect the expression or activity of endogenous RTN3 or RTN4, or combination of any of the above agent.

### DETAILED DESCRIPTION OF THE INVENTION

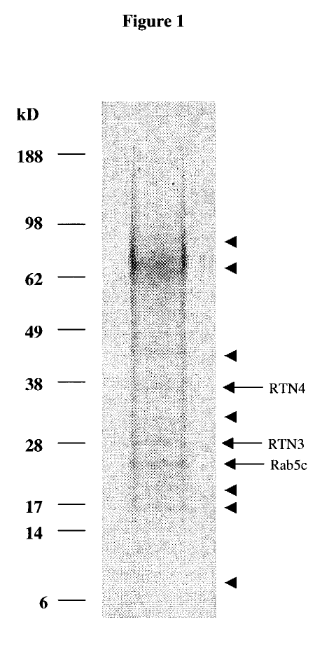

We have discovered that a class of proteins, previously not known to be associated with BACE, are important modulators of BACE activity. These are the proteins of the RTN family, specifically RTN3, RTN4, and rab5c. These BACE1 modulating proteins were identified from immunoprecipitation experiments from cells transfected with HA-tagged BACE1 with anti-HA antibody. The immunoprecipitated complex was resolved on 4-12% NuPage gel and stained with Colloidal Blue. After destained, the gels were sequenced. Immuno precipitation experiments revealed here showed in FIG. 1 that there were at least three proteins associated with the BACE1 in the immuno complex. We have determined that RTN3 proteins and RTN4 proteins interact with and modulate the activity of BACE1 either independently or in concert. We have also shown that increased expression of RTN3 and RTN4, together or independently, can lower or inhibit the activity of BACE1. The present invention relates to recombinant polypeptides that are derived from RTN3 sequence and possess one or more function or biological activities of RTN3 protein, polynucleotide sequences encoding the recombinant polypeptides, and method of making the recombinant polypeptides. The present invention further relates to assays that are developed based the novel finding that RTN3 proteins or RTN4 proteins modulate the activity of BACE1.

A. Polypeptides of the Invention

In one aspect, the present invention provides novel polypeptides (herein after polypeptides of the invention) which are derived from amino acid sequence of a human RTN3 and are functionally active, i.e., they are capable of displaying one or more known functional activities associated with a naturally occurring RTN3 protein. Such functional activities include, but are not limited to, ability to interact with BACE1 or modulate BACE1 activity, ability to bind (or compete with RTN3 for binding) to an anti-RTN3 antibody (antigenicity), and ability to generate antibody that binds to RTN3 protein (immunogenicity). The amino acid sequence of human RTN3 protein refers to the amino acid sequence of SEQ ID No. 2, which has 236 amino acids. The RTN3 amino acid sequence is disclosed in: E. F. Moreira, C. J. Jaworski, and I. R. Rodriguez, Cloning of a novel member of the reticulon gene family (RTN3): gene structure and chromosomal localization to 11q13. Genomics 58, 73-81 (1999).

Specifically, polypeptides of the invention include:

(a) an isolated polypeptide which comprises (i) a first polypeptide sequence consisting of about 85 to 97 consecutive amino acids of the N-terminus of SEQ ID No. 2, (ii) a second polypeptide sequence consisting of about 70 to 85 consecutive amino acids of the C-terminus of SEQ ID No. 2, and (iii) a third polypeptide sequence consisting of 0 to 55 amino acids, wherein the first polypeptide sequence is operably linked at its C-terminus to N-terminus of the second polypeptide sequence by the third polypeptide sequence;

(b) an isolated polypeptide which comprises (i) a first polypeptide sequence consisting of having at least 75, preferably 95% identity to about 97 consecutive amino acids of the N-terminus of SEQ ID No. 2, (ii) a second polypeptide sequence having 75%, preferably 95% identity to about 85 consecutive amino acids of the C-terminus of SEQ ID No. 2; and (iii) a third polypeptide sequence consisting of 0 to 55 amino acids, wherein the first polypeptide sequence is operably linked at its C-terminus to N-terminus of the second polypeptide sequence by the third polypeptide sequence.

(c) an isolated polypeptide which comprises (i) a first polypeptide sequence consisting of about 85 to 97 consecutive amino acids of the N-terminus of SEQ ID No. 2, (ii) a second polypeptide sequence consisting of about 70 to 85 consecutive amino acids of the C-terminus of SEQ ID No. 2, and (iii) and third polypeptide sequence consisting of 70 to 200 amino acids, wherein the first polypeptide sequence is operably linked at its C-terminus to N-terminus of the second polypeptide sequence by the third polypeptide sequence.

(d) an isolated polypeptide which comprises a (i) first polypeptide sequence consisting of having at least 75, preferably 95% identity to about 97 consecutive amino acids of the N-terminus of SEQ ID No. 2, (ii) a second polypeptide sequence having 75%, preferably 95% identity to about 85 consecutive amino acids of the C-terminus of SEQ ID No. 2; and (iii) a third polypeptide sequence consisting of about 70 to up to 200 amino acids, wherein the first polypeptide sequence is operably linked at its C-terminus to N-terminus of the second polypeptide sequence by the third polypeptide sequence.

(e) Variants of such polypeptides in (a) to (d) in which one or more amino acids, for instance from 1 to 15, from 1 to 10, from 1 to 5, from 1 to 3, or 1 amino acids are inserted, deleted, or substituted, in any combination, in either the first polypeptide sequence or the second polypeptide sequence, or both, of such polypeptides in (a) to (d).

Without whishing to be bound by theory, the first and second polypeptide sequences of the polypeptides of the invention are thought to be principally responsible for binding to and/or interacting with BACE1 and the third polypeptide sequence function is thought to help maintain a proper structural configuration of the polypeptide of the invention so that it can bind to and interact with BACE1. The length of the third polypeptide sequence is not critical so long it has either up to 60 amino acids or has between 70 to about 200 amino acids. It is preferred, however, that the length of the third polypeptide sequence is 1-60 amino acids, such as 10, 20, 30, 40, 50, 60 amino acids. In one embodiment, the isolated polypeptide of the invention consists of the first polypeptide sequence which is directly linked at the its C-terminus to the N-terminus of the second polypeptide sequence without intervening sequences between the first and second and polypeptide.

The amino acid sequence of the third polypeptide sequence may not be critical either. It is preferable, however, that the amino acid sequence of the third polypeptide sequence has at least 70%, 75%, 80%, 85%, 90%, or 95% identity to amino acids 97 to 160 of SEQ ID No. 2.

Variants of the polypeptides of the invention include insertion variants, wherein one or more amino acid residues are added to either the first polypeptide sequence, second polypeptide sequence, or both, of the of an aforementioned polypeptides. Insertions may be located at either or both termini of the polypeptide, or may be positioned within internal regions of the polypeptide sequence. Insertion variants with additional residues at either or both termini can include for example, fusion proteins and proteins including amino acid tags or labels. Insertion variants include polypeptides of the invention wherein one or more amino acid residues are added to a polypeptides sequence of the invention, or to a biologically active fragment thereof.

Various tag polypeptides and their respective antibodies are well known in the art. Examples include poly-histidine (poly-his) or poly-histidine-glycine (poly-his-gly) tags; the influenza HA tag polypeptide and its antibody 12CA5 [Field et al., Mol. Cell. Biol., 8:2159-2165 (1988)]; the c-myc tag and the 8F9, 3C7, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397(1990)]. In addition, a polypeptide of the invention can be tagged with enzymatic proteins such as peroxidase, GST and alkaline phosphatase.

The invention also provides deletion variants of polypeptides of the invention wherein one or more amino acid residues are removed from either the first polypeptide sequence or the second polypeptide sequence, or both, of an aforementioned polypeptides and the resulting variant retains at least one activity of the naturally occurring RTN3 protein. Deletions can be effected at either or both termini of the polypeptide, or within the amino acid sequence.

The present invention also includes include variants of the aforementioned polypeptides resulting from conservative amino acid substitutions, whereby a residue is substituted by another with like characteristics without substantially affecting the function of the polypeptide. Variant polypeptides include those wherein conservative substitutions have been introduced by modification of polynucleotides encoding polypeptides of the invention.

Method for producing a RTN3 or RTN4 polypeptide is known in the art. For example, a method of production of the RTN4 proteins by recombinant means are disclosed in WO 00/31235, WO 01/36631, and Tadzia GrandPre, et al. Nature, Vol. 403: 439-444 (2000). Polypeptides of the present invention can be prepared in any suitable manner, for instance by limited decomposition of RTN3 polypeptides, from genetically engineered host cells comprising expression systems, by chemical synthesis using, for instance, automated peptide synthesizers, or a combination of such methods. Means for preparing such polypeptides are well understood in the art.

B. Polynucleotides of the Invention

The present invention provides isolated polynucleotides (e.g., DNA sequences and RNA transcripts, both sense and complementary antisense strands, both single and double-stranded, including splice variants thereof) encoding a polypeptide of the invention. DNA polynucleotides of the invention include genomic DNA, cDNA, and DNA that has been chemically synthesized in whole or in part. The polynucleotides of the invention are derivatives of the coding region of the polynucleotides that encode a RTN3 protein. SEQ ID No. 1 is a cDNA sequence of the coding region that encodes a RTN3 protein, which is disclosed in: E. F. Moreira, C. J. Jaworski, and I. R. Rodriguez, Cloning of a novel member of the reticulon gene family (RTN3): gene structure and chromosomal localization to 11q13. Genomics 58, 73-81 (1999).

Specifically, the present invention provides polynucleotides which includes:

(a) an isolated polynucleotides which comprises (i) a first polynucleotide sequence consisting of about 255 to 291 consecutive bases of the 5′-terminus of SEQ ID No. 1, (ii) a second polynucleotide sequence consisting of about 210 to 255 consecutive bases of the 3′-terminus of SEQ ID No. 1, and (iii) a third polynucleotide sequence consisting of 0 to 165 consecutive bases, wherein the first polynucleotide sequence is operably linked at its 5′-terminus to 3′-terminus of the second polynucleotide sequence by the third polynucleotides sequence.

(b) an isolated polynucleotide which comprises (i) a first polynucleotide sequence having at least 75, preferably 95% identity to about 255 to 291 consecutive bases of the 5′-terminus of SEQ ID No. 1, (ii) a second polynucleotide sequence having 75%, preferably 95% identity to about 210 to 255 consecutive bases of the 3′-terminus of SEQ ID No. 1, and (iii) a third polynucleotide sequence consisting of either 0 to 165 consecutive bases or 210 to 600 consecutive bases, wherein the first polynucleotide sequence is operably linked at its 5′-terminus to 3′-terminus of the second polynucleotide sequence by the third polynucleotide sequence.

(c) an isolated polynucleotide having a polynucleotide sequence encoding a polypeptide sequence having at least 75%, preferably 95%, identity to a polypeptide sequence of the invention;

(d) an isolated polynucleotide encoding a polypeptide of the invention;

Polynucleotide of the present invention can be obtained from natural sources such as genomic DNA libraries or can be synthesized using well known and commercially available techniques. Polynucleotide of the present invention can also be prepared by a conventional cloning and screening techniques from a cDNA library from mRNA in cells of human tissues such as brain and spinal cord. A commercially available cDNA library derived from, for example, human brain can also be employed.

The cDNA can be amplified using suitable primers. Examples of primer pairs suitable for use in the PCR amplification include

(SEQ ID NO. 3)

5′-ATATATGGATCCCTCGCTCGCGTAGCCATGGC-3′ and

(SEQ ID NO. 4)

5′-ATATATGCGGCCGCGTTTCCATGTACTTATTC-3′.

For preparing a polypeptide of the invention that is fused to a tag, such as a His-Myc tag, another pair of PCR primers, such as

(SEQ ID NO. 5)

5′-AAAAAGGCAGAAGTACATGGAAACGCGGCCGC-3′ and

(SEQ ID NO. 6)

5′-TTCCATGTACTTTCTGCCTTTTTTTTGGCGATTCC-3′

may be used to remove the stop codon from the expression construct so that a polypeptide of the invention is fused to the tag in frame at the C-terminus.

C. Vectors, Host Cells, and Expression of the Invention

Polypeptides of the invention may be prepared by process well known in the art from genetically engineered host cells comprising expression systems. Accordingly, in a further aspect, the present invention provides (1) expression systems comprising a polynucleotide or polynucleotides of the invention, (2) host cells that are genetically engineered with such expression systems, and (3) production of polypeptides of the invention by recombinant techniques.

A great variety of expression systems can be used, for instance, plasmid and viral DNA vectors. Examples of mammalian expression systems suitable in the present invention includes pcDNA3.1 series (Invitrogen), pSVL (Pharmacia Biotech), pSVK ((Pharmacia Biotech), and pLP series (Clontech). The choice of a suitable expression vector for expression of polypeptides of the invention will of course depend upon the specific host cell to be used, and is within the skill of the ordinary artisan. The expressions system may contain an endogenous or exogenous expression control DNA sequence. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Commonly used promoter sequences and modifier sequences which may be used in the present invention include, but are not limited to, those derived from human cytomegalovirus (CMV), Adenovirus 2, Polyoma virus, and Simian virus 40 (SV40). Methods for the construction of mammalian expression vectors are disclosed, for example, in Okayama and Berg (Mol. Cell. Biol. 3:280 (1983)); Cosman et al. (Mol. Immunol. 23:935 (1986)); Cosman et al. (Nature 312:768 (1984)); EP-A-0367566; and WO 91/18982.

When polynucleotides of the present invention are used for the recombinant production of polypeptides of the present invention, the polynucleotide may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al., Proc Natl Acad Sci USA (1989) 86:821-824, or is an HA tag. In a preferred embodiment of the invention the mammalian expression pcDNA3/HisMyc vector is used. The polynucleotide may also contain non-coding 5′ and 3′ sequences, such as transcribed, non-translated sequences, splicing and polyadenylation signals, ribosome binding sites and sequences that stabilize mRNA. Examples of other commercially available expression vectors for use in prokaryotic hosts that comprise one or more phenotypic selectable marker genes include pSPORT vectors, pGEM vectors (Promega), pPROEX vectors (LTI, Bethesda, Md.), and Bluescript vectors (Stratagene).

The appropriate polynucleotide sequence may be inserted into an expression system by any of the techniques known in the art. Expression systems are preferably utilized for production of an encoded protein, but also may be utilized simply to amplify a polynucleotide sequence of the invention.

Suitable host cells for expression of the polypeptides of the invention include prokaryotes, yeast, and higher eukaryotic cells. Suitable prokaryotic hosts include but are not limited to bacteria of the genera Escherichia, Bacillus, and Salmonella, as well as members of the genera Pseudomonas, Streptomyces, and Staphylococcus.

Preferably, polynucleotides of the invention are cloned into a vector designed for expression in eukaryotic cells, rather than into a vector designed for expression in prokaryotic cells. Eukaryotic cells are sometimes preferred for expression of genes obtained from higher eukaryotes because the signals for synthesis, processing, and secretion of these proteins are usually recognized, whereas this is often not true for prokaryotic hosts (Ausubel, et al., ed., in Short Protocols in Molecular Biology, 2nd edition, John Wiley & Sons, publishers, pg.16-49, 1992.). Suitable eukaryotic hosts may include, but are not limited to, the following: insect cells, CHO, HEK-293, COS7, HeLa, IMR-32, SK-N-MC, and SK-N—SH.

Example of suitable yeast host cells include S. cerevisiae and P. pastoris. Yeast vectors will often contain an origin of replication sequence from a 2 micron yeast plasmid, an autonomously replicating sequence (ARS), a promoter region, sequences for polyadenylation, sequences for transcription termination, and a selectable marker gene. Vectors replicable in both yeast and E. coli (termed shuttle vectors) may also be used. In addition to the above-mentioned features of yeast vectors, a shuttle vector will also include sequences for replication and selection in E. coli.

For recombinant production, host cells can be genetically engineered to incorporate expression systems or portions thereof or polynucleotides of the invention. Introduction of a polynucleotide into the host cell can be effected by methods described in many standard laboratory manuals.

Polynucleotides of the invention may be introduced into the host cell as part of a circular plasmid, or as linear DNA comprising an isolated protein-coding region or a viral vector. Methods for introducing DNA into the host cell well known and routinely practiced in the art include transformation, transfection, electroporation, nuclear injection, or fusion with carriers such as liposomes, micelles, ghost cells, and protoplasts.

D. Compounds, Agents, and Methods of the Invention

The present invention further provides (1) methods to identify agents or compounds that modulate the expression of RTN3 or RTN4, (2) methods to identify agents or compounds that modulate the activity of RTN3 protein, RTN4 protein, or BACE1; (3) agents of compounds that modulate the expression or activity of RTN3 protein or RTN4 protein; (4) methods of modulating the activity of BACE1; and (5) method of treating CNS disorders. By “modulate” it is meant to increase, stimulate, decrease, magnify, mimic, disrupt, simulate, or otherwise change the level of activity of RTN3 protein, RTN4 protein, or BACE1, or change the level of expression of RT3 or RTN4, without regarding the specific underlying mechanisms by which a given agent asserts its effect. As used herein “RTN3 protein” or “RTN3 polypeptide” refers a gene product of RTN3 gene of human or non-human mammal such as mouse and bovine, such as a polypeptide of SEQ ID NO. 2. It also refers to variants and fragments of polypeptide of SEQ ID NO. 2 that substantially retain the BACE1 modulating function of a naturally occurring RTN3 protein, or to polypeptides that show at least 85%, preferably 95% identity to a polypeptide of SEQ ID NO. 2. As used herein “RTN4 protein” or “RTN4 polypeptide” refers to any of the three isoforms of the RTN4 gene products, namely RTN4-A protein, RTN4-B protein, and RTN4-C protein, which are also known as Nogo A protein, Nogo B protein, and Nogo C protein, respectively, of human and non-human mammal such as mouse and bovine. An amino acid sequence of human RTN4-A, RTN4-B, and RTB4-C is shown in SEQ ID NO. 7, SEQ ID NO. 8, and SEQ ID NO. 9. The term “RTN4 protein” also refers to variants and fragments of any RTN4 proteins that essentially retain the BACE1 modulating function of a naturally occurring RTN4 protein, and to polypeptides that show at least 85%, preferably 95%, identity to a polypeptide of SEQ ID NO. 7, SEQ ID NO. 8, or of SEQ ID NO. 9.

1. Methods to Identify Agents that Modulate Expression of RTN 3 or RTN4, or Activity of RTN3 Protein and RTN4 Protein

The present invention provides methods for identifying agents that modulate the expression or activity of RTN 3 protein or RTN 4 protein. The methods of the invention include both in vitro assays and in vivo assays.

An in vitro assay of the invention comprises steps of (1) contacting a test agent with a cell capable of expressing a RTN3 or a RTN4 and (2) measuring the level of activity or expression of RTN3 or RTN4 in the presence or absence of the test agent. Generally, to carry out the assay, the cells are maintained in a medium and under conditions suitable for these cells and the test agent is added to the medium. The cells that are exposed to the test agent are herein referred to as “treated cells.” Normally, a control cell culture is also prepared, which is the same cell culture maintained under similar conditions as the test cell culture except that the cells are not exposed to the test agent. After incubation of cells in the medium with or without the test agent for a predetermined period of time, the levels of expression or activity of the RTN3 or RTN4 are measured. The levels of expression or activity of the RTN3 or the RTN4 in the cells of the treated cell culture are compared with these in the control cells. Agents that modulate the expression or activity of RTN3 or RTN4 will be identified as causing a change, increase or decrease, in the express or activity of RTN3 or RTN4 in the treated cells relative to the control cells.

As used herein, the term “cell” refers to any mammalian cell lines, primary cell cultures, tissues, and organs that express or harbor the genes of RTN3 or RTN4. As used herein, the term “cell line” refers to a permanently established cell culture that will proliferate indefinitely given appropriate fresh medium and space. Examples of suitable cell line includes the COS-7, HEK293T, HeLa, CHO, IMR32, SK-N-MC, SH—N-AS, SK-N—SH, SK-N-DZ, SK-N-FI, F98, NCI-H187, NCI-H378, NCI-H526, LN-18, WER1-Rb-1, HepG2, MCP7, KB, A172, SH-SY5Y. All the above cell lines are commercially available. The culture methods and culture media for these cell lines are known in the art. The assay of the invention may also utilize primary cell cultures. As used herein the term “primary cell culture” refers to animal cells taken from a tissue source and their progeny grown in culture before subdivision and transfer to a subculture. Examples of the primary cell culture include liver cells from the liver and nerve cells from the nervous system of an animal. Tissues or organs removed from an animal can also be used in the assay. Normally the tissues or organs need to be prepared in small pieces or as homogenates in order to maximize the contact of the cells of the tissue with the test agent. Culture technologies for tissues, organs and cells are well known in the art and can be adopted readily for the assay of the invention. (See Paul, J. Cell and Tissue Culture, Fifth edition, Churchill Livingston Inc., NY, 1975; Kruse, P. F. and M. M. Patterson, eds. Tissue Culture Methods and Applications, Academic Press, NY, 1973.)

The test agents of the invention can be peptides, polypeptides, polynucleotides, antibodies, antibody fragments, small molecules, vitamin derivatives, or carbohydrates.

The amount of test agent that is brought into contact with the cells can vary and may be adjusted based on a variety of factors such as potency of the agent, density of the cells, and volume of the culture medium wherein the cells are maintained. The test agent can be added directly to the culture medium in the form of bulk drug or may be formulated in suitable carriers before being added to the culture medium. One or more test agents may be brought into contact with same cells, either consecutively or simultaneously, or otherwise.

Expression of a RTN3 or a RTN4 can be measured by standard methods for measuring gene expression known in the art, such as Northern blot, Western blot, ELISA, Tagman based PCR, competitive RT-PCR, competitive quantative RT-PCR (See protocol provided by Ambion, Inc), and RNA protection assay (Lee, J. J. and Costlow, N. A., A molecular titration assay to measure transcript prevalence levels. Method Enzymol. 152, 633-648, 1987). A typical indicator for the gene expression is mRNA transcribed from the target gene or a protein product of the target gene.

A Northern blot method for measuring RTN3 is disclosed by Moreira, at al. Genomics, 58, 73-81 (1999), in which the blot is probed with a 3′ untranslated RTN3-specific cDNA probe and the relative levels of expression are determined by normalizing the SYB green II stained 28S ribosomal RNA band to the signal generated by the probe using a STORM 860 instrument. An example of the RT-PCR method is also disclosed Moreira, at al. Genomics, 58, 73-81 (1999).

Western blot or ELISA can also be used to measure the levels of expression of RTN3 or RTN4 proteins. Peptide antibodies against RTN3 and RTN4 can be generated using standard methods known in the art and used to measure the protein levels of RTN3 or RTN4 in either cells expressing endogenous level of RTN3/RTN4 or in cells that were transfected with RTN3/RTN4 expressing constructs. Alternatively, RTN3 or RTN4 can be fused with a tag, such as myc, His, HA, Xpress, at either the C-terminus or N-terminus and the protein levels of the tagged RTN3 or RTN4 could be monitored by the specific anti-tag antibody.

Competitive RT-PCR is a method for quantifying mRNA. In this method, internal standard RNAs are added in a defined quantity to the RNA sample prior to the RT reaction. The resulting standard cDNA is coamplified with the same primers as the endogenous target sequence. Its PCR product is approximately 50 nucleotides smaller. This method allows measurement of small differences, as low as factor 2, in mRNA amount between RNA samples.

One of the target activities of RTN3 or RTN4 that may be measured in the assay of the invention is the function of the RTN3 or RTN4 to modulate an activity of BACE1, such as the APP processing activity of BACE1. This BACE1 modulating function of RTN3 or RTN4 may be measured indirectly by measuring the APP processing activity. The APP processing activity can be measured by methods known in the art, such as by measuring changes of A beta production in cells expressing both BACE1 and RTN3 or both BACE1 and RTN4. Thus, in a preferred embodiment, the activity of RTN3 or RTN4 is measured by measuring the A beta production in cells expressing both a BACE1 and a RTN3. In cells where the levels of activity of RTN3 or RTN4 is increased, the levels of secreted A beta in cultured medium is expected to be reduced in cells expressing endogenous levels of BACE1. Conversely, if the levels of activity of RTN3 or RTN4 is decreased, the levels of secreted A beta in cultured medium is expected to be increased. The levels of A beta can be measured by ELISA with antibody 6E10 as capturing antibody and Rb162 to detect A beta 40 and Rb165 to detect A beta 165. A method of measuring A beta production is disclosed in Yan, et al, Nature, 402, 533-537 (1999), which is incorporated herein be reference. Alternatively, A beta peptide can also be measured by different ELISA protocols according to the procedure described in many literatures or commercially available ELISA kits such as the one provided by Biosource International (Camarillo, Calif.).

The present invention also provides in vivo assays for identifying agents that modulate the expression or activity of RTN3 or RTN4. Such assays involve the use of animal models wherein a test agent is administered to the animal in appropriate doses, dose frequency, and durations. One or more groups of control animals, namely animals which do not receive the test agent, normally will also be used in the assay. Following the administration of the test, levels of expression or activity of RTN3 or RTN4 are measured in one or more tissues of the animals. The tissues can be sampled and processed according to standard methods known in the art. The level of expression or activity of RTN3 or RTN4 in the animals that received the test compound is compared with that in the control animals, that is animals that have not received the test compound. The species of animals that can be used in the assay is not critical. Any animals that express RTN3 or RTN4 or harbor a gene of RTN3 or RTN4 can be used in the assay. Examples of suitable animal species include rodents (rats, mice, hamsters, etc), rabbits, dogs, monkeys, pigs, cats, birds, or humans. Transgenic animals can also be used. Levels of expression or activity of RTN3 or RTN4 can be measured using methods described previously in this application or any other suitable methods known in the art.

2. Methods to Identify Agents that Modulate the Interactions between a RTN3 Protein and BACE1, or Between a RTN4 Protein and BACE1.

Another embodiment of the present invention provides methods for identifying agents that modulate (reduce or block or enhance, promote) the association of a RTN with a BACE1. Specifically, a BACE1 is mixed with a RTN protein, or a cellular extract containing a RTN, in the presence and absence of an agent to be tested. After mixing under conditions that allow association of the BACE1 with the RTN, the two mixtures are analyzed and compared to determine if the agent affected the association of the BACE1 with the RTN peptide. Agents that block or reduce the association of the BACE1 with the RTN will be identified as decreasing the amount of association present in the sample containing the tested agent. Agents that enhance or increase the association of the BACE1 with the RTN will be identified as increasing the amount of association present in the sample containing the tested agent. The RTN polypeptide used in the above assay can either be an isolated and fully characterized protein, such as a RTN3 or RTN4 or a RTN3 derivative of the invention, or can be a partially characterized protein that binds to BACE1 that has been identified as being present in a cellular extract. It will be apparent to one of ordinary skill in the art that so long as the RTN has been characterized by an identifiable property, e.g., molecular weight, the present assay can be used.

3. Methods to Identify Agents that Modulate BACE1 Activity

The present invention also provides methods for identifying agents that modulate the activity of BACE1. The methods of the invention utilize the level of expression or level of activity of RTN3 or RTN4 as indicators of the effect of a test agent on the BACE1 activity. Thus, the same methods that can be used to identify agents that modulate expression or activity or a RTN3 or RTN4 as described in the present application can be used to identify agents that modulate BACE1 activity. As used herein, an agent is said to modulate a BACE1 activity if the agent is capable of modulating the expression or activity of RTN3 or RTN4. Specifically, an agent is said to be a BACE1 inhibitor or antagonist if that agent is capable of causing an increase in, enhancement, or augmentation of expression or an activity of RTN3 or RTN4. Conversely, an agent is said to be a BACE1 stimulator or agonist if that agent is capable of causing a decrease or reduction in expression or activity of RTN3 or RTN4.

4. Agents that Modulate Expression of RTN3 or RTN4 or Modulate the Activity of RTN3, RTN4, or BACE1

The invention further provides agents that modulate the activity of a RTN3 or RTN4 polypeptide or a BACE1. Such compounds include those that can be identified by a person skilled in the art using the methods and procedures described herein above. The agents or compounds of the present invention can be, as examples, peptides, antibodies, antibody fragments, small molecules, vitamin derivatives, as well as carbohydrates. In a particular embodiment, the agent of the invention that modulates BACE1 activity is a RTN3 derivative (polypeptide) of the invention.

Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention is antibodies or fragments thereof that bind to a RTN3 or RTN4 polypeptide. Antibody agents can be obtained by immunization of suitable mammalian subjects with peptides, containing as antigenic regions, those portions of the protein intended to be targeted by the antibodies. This invention further provides peptide mimetics of a RTN3 protein, a RTN4 protein, or a polypeptide of the invention. As used herein, “peptide mimetics” refer to (1) peptide-containing molecules that either mimic elements of protein secondary structure of RTN3, RTN4, or of a polypeptide of the invention, or mimic biochemical property or pharmacological activity of RTN3 or RTN4, including the BACE1 activity modulating property of RTN3, RTN4, or of a polypeptide of the invention, or (2) non-peptide compounds that are properties analogous with properties analogous to those of the template peptide. Peptide mimetics may have significant advantages over naturally-occurring peptides, including, for example: more economical production, greater chemical stability, enhanced pharmacological properties (half-life, absorption, potency, efficacy, etc.), altered specificity (e.g., a broad-spectrum of biological activities), reduced antigenicity, and others. Peptide mimetics of RTN3, RTN4 and peptides of the invention can be constructed by structure-based drug design known in the art. For general information on peptide mimetics, see, for example; Jones, (1992) Amino Acid and Peptide Synthesis, Oxford University Press; Jung, (1997) Combinatorial Peptide and Nonpeptide Libraries: A Handbook, John Wiley; Bodanszky et al., (1993) Peptide Chemistry—A Practical Textbook, Springer Verlag.

5. Methods of Modulating BACE1 Activity and Treating Disorders

As described previously, Applicant's discovery showed that RTN3 proteins or RTN4 proteins modulate BACE1 activity. Specifically, Applicants found that increased expression or activity of RTN3 or RTN4 would decrease BACE1 activity. Accordingly, in a further aspect, the invention provides a method of decreasing BACE1 activity in cells of a mammal comprising administering to such mammal one of more agents selected from the group consisting of

(a) a RTN3 polypeptide

(b) a RTN4 polypeptide;

(c) a polypeptide of the invention;

(d) a RTN3 mimic;

(e) a RTN4 mimic;

(f) an agent that increases expression of RTN3

(g) an agent that increases expression of RTN4

(h) an agent that increases activity of RTN3 proteins and/or binding affinity to BACE1;

(i) an agent that increases activity of RTN4 proteins and/or binding affinity to BACE 1, and wherein the amount of the agent is effective to decrease the activity of BACE1.

As described previously, BACE1 activity is found to be closely associated with the formation of A beta peptides). Increased production of A beta peptides causes the amyloid deposition 1) in the hippocampus and frontal cortex that contributes to the pathogenesis of Alzheimer's disease, 2) in the vascular area that contributes to the pathogenesis of cerebral amyloid angiopathy (CAA), 3) vacuolated muscle fibers that may contribute to Sporadic inclusion-body myositis (IBM), the most common progressive muscle disease of older individuals. Thus, agents that decrease BACE1 activity, which in turn decreases A beta production, may be useful in treating disorders that are associated with A beta deposition. Accordingly, the invention further provides a method of treating or delaying the onset of disorders that are associated with A beta deposition in a mammal comprising administering an effective amount of one or more agents selected from the group consisting of:

(a) a RTN3 polypeptide

(b) a RTN4 polypeptide;

(c) a polypeptide of the invention;

(d) a RTN3 mimic;

(e) a RTN4 mimic;

(f) an agent that increases expression of RTN3;

(g) an agent that increases expression of RTN4;

(h) an agent that increases activity of RTN3 proteins and/or binding affinity to BACE1; and

(i) an agent that increases activity of RTN4 proteins and/or binding affinity to BACE1.

Examples of the disorders contemplated in the invention include Alzheimer's disease, Cerebral Amyloid Angiopathy (CAA), 3), and Sporadic Inclusion-Body Myositis (IBM).

The agents for modulating BACE 1 activity or treating disorders of the present invention can be provided alone, or in combination with other therapeutic or diagnostic agents. In certain preferred embodiments, the compounds of this invention may be co-administered along with other compounds typically prescribed for these conditions according to generally accepted medical practice, such as ARICEPT® (donepezil HCl) from Pfizer/Eisa, Reminyl® (galantamine HBr) from Janssen, Liptor, Vioxx, and cerebrax.

The agents of the present invention can be administered via any suitable route, such as parenteral, subcutaneous, intravenous, intramuscular, intraperitoneal, transdermal, or buccal routes.

The dosage administered will be dependent upon the age, health, and weight of the recipient, kind of concurrent treatment, if any, frequency of treatment, and the nature of the effect desired.

### Definitions

The definitions and explanations below are for the terms as used throughout this entire document including both specification and the claims.

“Polynucleotide” generally refers to any polyribonucleotide (RNA) or polydeoxribonucleotide (DNA), which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “polynucleotide” also embraces relatively short polynucleotides, often referred to as oligonucleotides.

“Polypeptide” refers to any polypeptide comprising two or more amino acids joined to each other by peptide bonds or modified peptide bonds. “polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications.

“Isolated” means altered “by the hand of man” from its natural state, i.e., if it occurs in nature, it has been changed or removed from its original environment, or both. For example, a polynucleotide or a polypeptide naturally present in a living organism is not “Isolated,” but the same polynucleotide or polypeptide separated from the coexisting materials of its natural state is “isolated.” Moreover, a polynucleotide or polypeptide that is introduced into an organism by transformation, genetic manipulation or by any other recombinant method is “isolated” even if it is still present in said organism, which organism may be living or non-living. As used herein therefore, by way of example only, a transgenic animal or a recombinant cell line constructed with a polynucleotide of the invention makes use of the “isolated” nucleic acid.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared. For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so called local alignment), that is more suitable for sequences of unequal length.

“Fusion protein” refers to a protein encoded by two, often unrelated, fused genes or fragments thereof.

“Host cell” is a cell which has been transformed or transfected, or is capable of transformation or transfection by an exogenous polynucleotide sequence.

“Amyloid” refers to a form of aggregated protein.

“Amyloidosis” refers to any disease characterized by the extracellular accumulation of amyloid in various organs and tissues of the body.

### EXAMPLES

### Example 1

### Demonstration of Association of RTN3 with BACE1

To demonstrate the association of RTN3 with BACE1, we performed immunoprecipitation experiments from HA-tagged BACE1 transfected cells using anti-HA antibody. HEK 293 cells were obtained from grown and maintained at 37° C. in a humidified, CO₂ controlled atmosphere in Dulbecco's modified Eagle's medium (DMEM) supplemented with 10% FBS, 50 IU/ml penicillin, 50 μg/ml streptomycin and glutamine. This cell line was used to generate a stable cell line expressing HA-tagged BACE1 under the selection of hygromycin B. Transfections were performed using the Lipofectaime 2000© reagent. A total of 20 μg of DNA were transfected into 10 cm dishes with 80 μl of Lipofectamine 2000® reagent. DNA and lipofectamine solutions were mixed in a total of 2 ml Opti-MEM media for 15 min and then added the mixture to each dish containing 8 ml of antibiotic free DMEM. Colloidal Blue stained SDS-PAGE gel of immunoprecipitated complex displayed one intense band corresponding to mature BACE1 near 65 kD (FIG. 1). Mass spectroscopy based sequencing confirmed it as BACE1. Sequencing of other smaller bands showed that majority of the bands were corresponding to the BACE1 fragments. Several bands in the range of 17-38 kD were identified as one small GTP-binding protein rab5c, RTN 3 and RTN4.

To confirm that RTN 3 is associated with BACE1, we cloned full length RTN 3 from a human brain library by PCR amplification. A pair of primers (5′-ATATATGGATCCCTCGCTCGCGTAGCCATGGC-3′ and 5′-ATATATGCGGCCGCGTTTCCATGTACTTATTC-3′) was used to amplify the entire coding region of RTN 3 from a human brain cDNA library. The PCR fragment was first digested with restriction enzymes Bam HI and Not I and then inserted into a pretreated vector (pCDNA3.1/hismyc). The expression construct was sequenced on both strands to ensure the fidelity. Another pair of PCR primers (5′-AAAAAGGCAGAAGTACATGGAAACGCGGCCGC-3′ and TTCCATGTACTTTCTGCCTTTTTTTTGGCGATTCC-3′) was used to remove the stop codon from the above expression construct so that RTN 3 was fused to His-Myc tag in frame at the C-terminus. The coding region of RTN 3 was inserted into a mammalian expression pCDNA3/HisMyc vector. Transfection of RTN 3 in cells expressing HA-tagged BACE produce a major band 25 kD. To replicate RTN 3 binding to BACE1, we performed immunoprecipitation of the transfected cells with an anti-HA antibody followed by Western analysis of immunoprecipitated complex with anti-myc antibody. We observed RTN 3 in cells expressing RTN3, but not in vector expressing cells. This result was consistent with the identification of endogenous RTN 3 in the immunoprecipitated complex by anti-HA tagged BACE1 shown in FIG. 1. To further confirm this association, we reciprocally immunoiprecipitated cell extracts with an anti-myc antibody and found that BACE 1 was indeed in the complex pulled-down against myc-tagged RTN 3. Thus, it is concluded that RTN 3 forms tight complex with BACE1 in cells.

### Example 2

### Demonstration of Modulation of BACE1 Activity by RTN3

The influence of RTN3 protein on the activity of BACE1 on Aβ peptide release in cells was evaluated by measuring levels of selected Aβ peptides in the conditioned medium from those cells transfected with either vector control or RTN 3 using ELISA. We found that the levels of the Aβ peptide release are affected the levels of RTN3 expression.

What is calimed is:

1. An isolated polypeptide selected from the group consisting of
(a) an isolated polypeptide which comprises (i) a first polypeptide sequence consisting of about 85 to 97 consecutive amino acids of the N-terminus of SEQ ID No. 2, (ii) a second polypeptide sequence consisting of about 70 to 85 consecutive amino acids of the C-terminus of SEQ ID No. 2, and (iii) a third polypeptide sequence consisting of 0 to 55 amino acids, wherein the first polypeptide sequence is operably linked at its C-terminus to N-terminus of the second polypeptide sequence by the third polypeptide sequence;
(b) an isolated polypeptide which comprises (i) a first polypeptide sequence consisting of having at least 75, preferably 95% identity to about 97 consecutive amino acids of the N-terminus of SEQ ID No. 2, (ii) a second polypeptide sequence having 75%, preferably 95% identity to about 85 consecutive amino acids of the C-terminus of SEQ ID No. 2; and (iii) a third polypeptide sequence consisting of 0 to 55 amino acids, wherein the first polypeptide sequence is operably linked at its C-terminus to N-terminus of the second polypeptide sequence by the third polypeptide sequence;
(c) an isolated polypeptide which comprises (i) a first polypeptide sequence consisting of about 85 to 97 consecutive amino acids of the N-terminus of SEQ ID No. 2, (ii) a second polypeptide sequence consisting of about 70 to 85 consecutive amino acids of the C-terminus of SEQ ID No. 2, and (iii) and third polypeptide sequence consisting of 70 to 200 amino acids, wherein the first polypeptide sequence is operably linked at its C-terminus to N-terminus of the second polypeptide sequence by the third polypeptide sequence;
(d) an isolated polypeptide which comprises a (i) first polypeptide sequence consisting of having at least 75, preferably 95% identity to about 97 consecutive amino acids of the N-terminus of SEQ ID No. 2, (ii) a second polypeptide sequence having 75%, preferably 95% identity to about 85 consecutive amino acids of the C-terminus of SEQ ID No. 2; and (iii) a third polypeptide sequence consisting of about 70 to up to 200 amino acids, wherein the first polypeptide sequence is operably linked at its C-terminus to N-terminus of the second polypeptide sequence by the third polypeptide sequence; and
(e) Variants of such polypeptides in (a) to (d) in which one or more amino acids, for instance from 1 to 15, from 1 to 10, from 1 to 5, from 1 to 3, or 1 amino acids are inserted, deleted, or substituted, in any combination, in either the first polypeptide sequence or the second polypeptide sequence, or both, of such polypeptides in (a) to (d).

2. An isolated polynucleotide selected from the group consisting of:
(a) an isolated polynucleotides which comprises (i) a first polynucleotide sequence consisting of about 255 to 291 consecutive bases of the 5′-terminus of SEQ ID No. 1, (ii) a second polynucleotide sequence consisting of about 210 to 255 consecutive bases of the 3′-terminus of SEQ ID No. 1, and (iii) a third polynucleotide sequence consisting of 0 to 165 consecutive bases, wherein the first polynucleotide sequence is operably linked at its 5′-terminus to 3′-terminus of the second polynucleotide sequence by the third polynucleotides sequence;
(b) an isolated polynucleotide which comprises (i) a first polynucleotide sequence having at least 75, preferably 95% identity to about 255 to 291 consecutive bases of the 5′-terminus of SEQ ID No. 1, (ii) a second polynucleotide sequence having 75%, preferably 95% identity to about 210 to 255 consecutive bases of the 3′-terminus of SEQ ID No. 1, and (iii) a third polynucleotide sequence consisting of either 0 to 165 consecutive bases or 210 to 600 consecutive bases, wherein the first polynucleotide sequence is operably linked at its 5′-terminus to 3′-terminus of the second polynucleotide sequence by the third polynucleotide sequence;
(c) an isolated polynucleotide having a polynucleotide sequence encoding a polypeptide sequence having at least 75%, preferably 95%, identity to a polypeptide sequence of claim 1; and
(d) an isolated polynucleotide encoding a polypeptide of claim 1.

3. The isolated polynucleotide of claim 2, wherein said polynucleotide is operably linked to one or more expression control elements.

4. A vector comprising a polynucleotide of claim 2 or 3.

5. A host cell transformed to contain a polynucleotide of claim 2 or 3.

6. A host cell comprising a vector of claim 4.

7. A method for producing a polypeptide of claim 2 comprising the step of culturing a host cell transformed with the polynucleotide of claim 2 or 3 under conditions in which the protein encoded by said nucleic acid molecule is expressed.

8. A fusion protein comprising a polypeptide of claim 1.

9. A method of identifying an agent that modulates RTN3 expression in a cell comprising the step of:
(a) contacting a test agent with a cell expressing or capable of expressing a RTN3 polypeptide and
(c) comparing the levels of RTN3 expression in the presence and absence of the test agent, wherein a difference is indicative of the test agent that modulates RTN3 expression.

10. The method of claim 9 wherein the cell is selected from a cell line or a primary cell culture.

11. The method of claim 9 wherein the cell is a cell line.

12. The method of claim 11 wherein the cell line is selected from the group consisting of COS-7, HEK293T, HeLa, CHO, IMR32, SK-N-MC, SH-N-AS, SK-N-SH, SK-N-DZ, SK-N-FI, F98, NCI-H187, NCI-H378, NC1-H526, LN-18, WER1-Rb-1, HepG2, MCP7, KB, A172, and SH-SY5Y.

13. A method of identifying an agent that modulates RTN3 expression cells of an animal comprising the step of:
(a) administering a test agent to the animal expressing or capable of expressing a RTN3 polypeptide,
(b) sampling a tissue from the animal, and
(c) comparing the levels of RTN3 expression in the tissue of the animal with and without the administration of the test agent, wherein a difference is indicative of the test agent that modulates RTN3 expression.

14. The method of claim 13 wherein the animal is a mammal.

15. A method of identifying an agent that modulates RTN3 protein activity in a cell comprising the step of:
(a) contacting a test agent with a cell comprising a RTN3 protein and
(b) comparing the levels of RTN3 protein activity in the presence and absence of the test agent, wherein a difference is indicative of the test agent that modulates RTN3 protein activity.

16. The method of claim 15 wherein the cell is selected from a cell line or a primary cell culture.

17. The method of claim 16 wherein the cell is a cell line.

18. The method of claim 17 wherein the cell line is selected from the group consisting of COS-7, HEK293T, HeLa, CHO, IMR32, SK-N-MC, SH-N-AS, SK-N-SH, SK-N-DZ, SK-N-F1, F98, NCI-H187, NCI-H378, NCI-H526, LN-18, WER1-Rb-1, HepG2, MCP7, KB, A172, and SH-SY5Y.

19. A method of identifying an agent that modulates RTN3 protein activity in cells of an animal comprising the step of:
(a) administering a test agent to the animal the cells of which comprises a RTN3 protein,
(b) sampling a tissue from the animal, and
(c) comparing the levels of RTN3 protein activity in the tissue of the animal with and without the administration of the test agent, wherein a difference is indicative of the test agent that modulates RTN3 protein activity.

20. The method of claim 19 wherein the animal is a mammal.

21. A method of identifying agents that modulate the association of a reticulon (RTN) protein with a BACE1 comprising the step of
(a) contacting a BACE1 with a RTN protein, a RTN protein derivative, or a cellular extract containing a RTN protein in the presence and absence of the test agent, and
(b) comparing the association of the BACE1 with the RTN protein in the presence and absence of the test agent; wherein a difference is indicative of the test agent that modulates the association.

22. The method of claim 21 wherein the RTN protein is a RTN3 protein.

23. A method of identifying an agent that modulates BACE1 activity comprising the steps of:
(a) providing a cell expressing a RTN3 protein;
(b) contacting the cell with a test agent; and
(c) detecting the level of expression or activity of RTN3 protein in the presence and absence of the test agent, wherein a difference is indicative of the test agent that modulates BACE1 activity.

24. A method of identifying an agent that modulate beta amyloid peptide production comprising the steps of:
(a) providing a cell expressing a RTN3 protein;
(b) contacting the cell with a test agent; and
(c) detecting the level of expression or activity of RTN3 protein in the presence and absence of the test agent, wherein a difference is indicative of the test agent that modulates beta amyloid peptide production.

25. A method of decreasing BACE1 activity in cells of a human or non-human animal comprising administering to the animal an effective amount of one or more agents selected from the group consisting of:
(a) a RTN3 polypeptide
(b) a RTN4 polypeptide;
(c) a polypeptide of claim 1;
(d) a RTN3 mimic;
(e) a RTN4 mimic;
(f) an agent that increases expression of RTN3 polypeptide;
(g) an agent that increases expression of RTN4 polypeptide;
(h) an agent that increases activity of RTN3 polypeptide; and
(i) an agent that increases activity of RTN4 polypeptide.

26. A method of treating or delaying the onset of disorders that are associated with beta amyloid peptide deposition in a mammal comprising administering to the mammal an effective amount of one or more agents selected from the group consisting of:
(a) a RTN3 polypeptide
(b) a RTN4 polypeptide;
(c) a polypeptide of the invention;
(d) a mimic of a RTN3 polypeptide;
(e) a mimic of a RTN4 polypeptide;
(f) an agent that increases expression of RTN3 polypeptide;
(g) an agent that increases expression of RTN4 polypeptide;
(h) an agent that increases activity of RTN3 polypeptide; and
(i) an agent that increases activity of RTN4 polypeptide.

27. The method of claim 26 wherein the mammal is a human.

28. The method of 27 wherein the disorder is selected from the group consisting of Alzheimer's disease, Cerebral Amyloid Angiopathy, and Sporadic Inclusion-Body Myositis.

29. The method of 28 wherein the disorder is Alzheimer's disease.

30. A method of identifying an agent that modulates RTN4 expression in a cell comprising the step of:
(a) contacting a test agent with a cell expressing or capable of expressing a RTN4 polypeptide and
(b) comparing the levels of RTN4 expression in the presence and absence of the test agent, wherein a difference is indicative of the test agent that modulates RTN4 expression.

31. The method of claim 9 wherein the cell is selected from a cell line or a primary cell culture.

32. The method of claim 9 wherein the cell is a cell line.

33. The method of claim 11 wherein the cell line is selected from the group consisting of COS-7, HEK293T, HeLa, CHO, IMR32, SK-N-MC, SH-N-AS, SK-N-SH, SK-N-DZ, SK-N-FI, F98, NCI-H187, NCI-H378, NCI-H526, LN-18, WER1-Rb-1, HepG2, MCP7, KB, A172, and SH-SY5Y.

34. A method of identifying an agent that modulates RTN4 expression cells of an animal comprising the step of:
(a) administering a test agent to the animal expressing or capable of expressing a RTN4 polypeptide,
(b) sampling a tissue from the animal, and
(c) comparing the levels of RTN4 expression in the tissue of the animal with and without the administration of the test agent, wherein a difference is indicative of the test agent that modulates RTN4 expression.

35. The method of claim 34 wherein the animal is a mammal.

36. A method of identifying an agent that modulates RTN4 protein activity in a cell comprising the step of:
(a) contacting a test agent with a cell comprising a RTN4 protein and
(b) comparing the levels of RTN4 protein activity in the presence and absence of the test agent, wherein a difference is indicative of the test agent that modulates RTN4 protein activity.

37. The method of claim 36 wherein the cell is selected from a cell line or a primary cell culture.

38. The method of claim 37 wherein the cell is a cell line.

39. The method of claim 37 wherein the cell line is selected from the group consisting of COS-7, HEK293T, HeLa, CHO, IMR32, SK-N-MC, SH-N-AS, SK-N-SH, SK-N-DZ, SK-N-F1, F98, NCI-H187, NCI-H378, NCI-H526, LN-18, WER1-Rb-1, HepG2, MCP7, KB, A172, and SH-SY5Y.

40. A method of identifying an agent that modulates RTN4 protein activity in cells of an animal comprising the step of:
(a) administering a test agent to the animal the cells of which comprises a RTN4 protein,
(b) sampling a tissue from the animal, and
(c) comparing the levels of RTN4 protein activity in the tissue of the animal with and without the administration of the test agent, wherein a difference is indicative of the test agent that modulates RTN4 protein activity.

41. The method of claim 40 wherein the animal is a mammal.

42. A method of identifying an agent that modulates BACE1 activity comprising the steps of:
(a) providing a cell expressing a RTN4 protein;
(b) contacting the cell with a test agent; and
(c) detecting the level of expression or activity of the RTN4 protein in the presence and absence of the test agent, wherein a difference is indicative of the test agent that modulates BACE1 activity.

43. A method of identifying an agent that modulate beta amyloid peptide production comprising the steps of:
(a) providing a cell expressing a RTN4 protein;
(b) contacting the cell with a test agent; and
(c) detecting the level of expression or activity of RTN4 protein in the presence and absence of the test agent, wherein a difference is indicative of the test agent that modulates beta amyloid peptide production.

44. The method of any of claims 23, 24, 42, and 42 wherein the cell is a cell line.

45. The method of claim 44 wherein the cell line is selected from the group consisting of COS-7, HEK293T, HeLa, CHO, IMR32, SK-N-MC, SH-N-AS, SK-N-SH, SK-N-DZ, SK-N-F1, F98, NCI-H187, NCI-H378, NC1-H526, LN-18, WER1-Rb-1, HepG2, MCP7, KB, A172, and SH-SY5Y.

46. A method of treating amyloidosis in a human or non-human animal subject, said method comprising administering to said subject an effective amount of one or more agents selected from the group consisting of:
(a) a RTN3 polypeptide
(b) a RTN4 polypeptide;
(c) a polypeptide of claim 1;
(d) a mimic of a RTN3 polypeptide;
(e) a mimic of a RTN4 polypeptide;
(f) an agent that increases expression of RTN3 polypeptide;
(g) an agent that increases expression of RTN4 polypeptide;
(h) an agent that increases activity of RTN3 polypeptide; and
(i) an agent that increases activity of RTN4 polypeptide.

47. The method of any one of claims 25, 26, and 46 wherein the RTN3 polypeptide is a polypeptide of SEQ ID NO. 2.

48. The method of any one of claims 25, 26, and 46 wherein the RTN3 polypeptide is a variant or fragment of a polypeptide of SEQ ID NO. 2.

49. The method of any one of claims 25, 26, and 46 wherein the RTN3 polypeptide is a polypeptide having at least 85% identity with a polypeptide of SEQ ID NO. 2.

50. The method of claim 49 wherein the RTN3 polypeptide is a polypeptide having at least 95% identity with a polypeptide of SEQ ID NO. 2.

51. The method of any one of claims 25, 26, and 46 wherein the RTN4 polypeptide is a RTN4-A polypeptide, RTN4-B polypeptide, or RTN4-C polypeptide.

52. The method of any one of claims 25, 26, and 46 wherein the RTN4 polypeptide is a polypeptide of SEQ ID NO. 7, SEQ ID NO. 8, or SEQ ID NO. 9.

53. The method of any one of claims 25, 26, and 46 wherein the RTN4 polypeptide is a variant or fragment of a polypeptide of SEQ ID NO. 7, SEQ ID NO. 8, or SEQ ID NO. 9,

54. The method of any one of claims 25, 26, and 46 wherein the RTN4 polypeptide is a polypeptide of SEQ ID NO. 7, SEQ ID NO. 8, or SEQ ID NO. 9.

55. The method of any one of claims 25, 26, and 46 wherein the RTN4 polypeptide is a polypeptide having at least 85% identity with a polypeptide of SEQ ID NO. 7, SEQ ID NO. 8, or SEQ ID NO. 9.

56. The method of claim 55 wherein the RTN4 polypeptide is a polypeptide having at least 95% identity with a polypeptide of SEQ ID NO. 7, SEQ ID NO. 8, or SEQ ID NO. 9.
